(19) United States
(12) Patent Application Publication
(10) Pub. No.: US 2016/0367672 A1
(43) Pub. Date: Dec. 22, 2016

(54) METHODS AND SYSTEMS FOR TREATING TUMORS

(71) Applicant: UNIVERSITY OF SOUTHERN CALIFORNIA, Los Angeles, CA (US)

(72) Inventors: Thomas C. CHEN, La Cañada Flintridge, CA (US); Florence M. HOFMAN, Venice, CA (US); Tiago A.R.T.S. SANTOS, Penafiel (PT)

(73) Assignee: University of Southern California, Los Angeles, CA (US)

(21) Appl. No.: 15/122,352

(22) PCT Filed: Mar. 3, 2015
(86) PCT No.: PCT/US2015/018510

§ 371 Date: Aug. 29, 2016

Related U.S. Application Data
(60) Provisional application No. 61/947,287, filed on Mar. 3, 2014.

(51) Int. Cl. A61K 41/00; A61N 5/06; A61B 18/04; A61K 45/06

(52) U.S. Cl. CPC A61K 41/0052; A61K 45/06; A61N 5/0601; A61N 5/0625; A61B 18/04; A61N 2005/0612; A61N 2005/0659; A61N 2005/067

(57) ABSTRACT

The invention relates methods and systems of using a nanoparticle and near infrared radiation to treat, prevent, reduce the likelihood of having, reduce the severity of and/or slow the progression of a condition in a subject. The invention also relates to compositions comprising a nanoparticle. Conditions treatable with the methods systems and compositions include but are not limited to various tumors.

### FIELD OF THE INVENTION

The invention relates to methods and systems for treating a condition with a nanotube and near infrared radiation. The condition includes but is not limited to various tumors, particularly chemoresistant irradiated brain tumors.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Glioblastoma multiforme (GBM), World Health Organization (WHO) grade IV, is the most aggressive type of primary brain tumor. Median survival is only 12-15 months and 5-year survival is less than 5% regardless of treatment. Standard of care consists of surgery, followed by radiation and chemotherapy, usually temozolomide (TMZ). These therapies have significant side effects (for example, severe off-target effects to healthy tissues), cause the selection of treatment-resistant cancer cell clones responsible for tumor recurrence, and routinely result in the recurrence of drug resistant tumors. Unfortunately upon recurrence, patients have very limited treatment options. Current gamma radiation therapy is limited because it usually can only be applied once. Once a certain amount of radiation has been applied to the tumor field, additional radiation runs the risk of inducing damage to adjacent brain, resulting in cognitive or neurological deficits or radiation necrosis. Chemotherapy options are currently limited as GBMs rapidly develop chemoresistance, and the choice of chemotherapy is often limited by the blood brain barrier (BBB). Chemotherapy with bevacizumab is often used as second line therapy. Further ionizing radiation is generally not recommended, although radiosurgery boost has been used in some select situations.

Photothermal treatment is an alternative therapy which induces cytotoxicity to drug sensitive and resistant tumor cells. This therapy involves the induction of hyperthermia, defined as temperatures above 40° C. This therapeutic intervention causes irreparable cell damage to tumor cells, while causing some damage to but generally sparing normal cells. Currently, radiofrequency ablation is used in the clinic to treat mainly kidney, lung and liver tumors.

The main problem with hyperthermia is the difficulty in specifically targeting cell populations for destruction. As a result, hyperthermia has traditionally been delivered to the center of the tumor using a heat emitting device (i.e. laser) directed stereotactically into the malignant glioma. Presently, two hyperthermic therapies for brain tumor are beginning clinical trials. Monteris Medical, Incorporated is currently testing the FDA cleared NeuroBlade™. This is an MRI-guided system that enables laser ablation of brain tumors using a needle-like probe similarly to radiofrequency abalation. BioTex, Inc. is testing Visualase® Thermal Therapy System, a therapy identical to the one described above.

These methods involve the use of a physical probe (needle-like) to elevate tumor temperature. Limitations of this procedure include unexplored feasibility of the technique for the treatment of brain tumors, an uneven tumor heating due to the single point source of hyperthermia, as well as reported tumor seeding and metastasis along the probe tract. Moreover, every new treatment involves an invasive penetration into the brain parenchyma to insert the probe into the tumor. Thus, this type of treatment can only be performed once, and future treatments would require another invasive procedure into the brain. Hence, despite its local efficacy, radiofrequency ablation and similar treatments have been limited by the failure to generate tumor-specific heating in a minimally invasive manner.

In this invention, we demonstrate that the combination of carbon nanotubes (CNTs) and near infrared radiation (NIR) is an effective photothermal therapy that selectively affects both drug-sensitive and -resistant glioma cells and tumor initiating glioma cancer stem cells, while sparing normal cells. Furthermore, these studies demonstrate that this therapy is effective in vivo for drug-resistant tumors without significant pathology to neighboring normal control tissues. Therefore, we provide methods, systems and composition for using a nanoparticle and NIR to treat various tumors, including but not limited to brain tumors.

### SUMMARY OF THE INVENTION

Various embodiments of the present invention provide a method of treating, preventing, reducing the likelihood of having, reducing the severity of and/or slowing the progression of a condition in a subject. In various embodiments, the subject is a human. In variou embodiments, the conditoin is a tumor or cancer. In certain embodiments, the condition is brain tumor, glioma, glioblastoma, and/or glioblastoma multiforme (GBM). The method may comprise or may consist of: providing a nanoparticle; administering a therapeutically effective amount of the nanoparticle to the subject; applying near infrared radiation (NIR) to the subject to induce hyperthermia, thereby treating, preventing, reducing the likelihood of having, reducing the severity of and/or slowing the progression of the condition in the subject.

In various embodiments, the method can further comprisea providing a chemotherapeutic agent and administering a therapeutically effective amount of the chemotherapeutic agent to the subject. In some embodiments, the nanoparticle and the chemotherapeutic agent are adminstered together. In other embodiments, the nanoparticle and the chemotherapeutic agent are adminstered separately. In various embodiments, the nanoparticle is adminstered before, during or after administeirng the chemotherapeutic agent.

In various embodiments, the nanoparticle is provided in a pharmaceutical composition. In various embodiments, the chemotherapeutic agent is provided in a pharmaceutical composition. In various embodiments, the nanoparticle and the chemotherapeutic agent are provided in one pharmaceutical composition. In various embodiments, the nanoparticle and the chemotherapeutic agent are provided in separate pharmaceutical compositions that may be administered together or individually. In one embodiment, the nanoparticle is conjugated to the chemotherapeutic agent. In another embodiment, the nanoparticle is loaded with the chemotherapeutic agent. In some embodiments, the nanoparticle is not conjugated to or loaded with the chemotherapeutic agent. In accordance with the present invention, a pharmaceutical composition described herein can further comprise a pharmaceutically acceptable excipient and/or a pharmaceutically acceptable carrier.

In various embodiments, the nanoparticle is a nanotube, nanorod, nanoshell, nanocage, nanosphere, nanofiber, or nanowire, or a combination thereof. In various embodiments, the nanoparticle is made of carbon, gold, selenium, copper, platinum, or a combination thereof. In some embodiments, the nanoparticle is administered intratumorally, intracranially, intraventricularly, intrathecally, epidurally, intradurally, intravascularly, intravenously, intraarterially, intramuscularly, subcutaneously, intraperitoneally, orally, intranasally, or via inhalation. In some embodiments, the nanoparticle is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 ng per mm³ tumor. In some embodiments, the nanoparticle is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 μg. In some embodiments, the nanoparticle is administered once, twice, three or more times.

In some embodiments, NIR is applied at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 W/cm². In some embodiments, NIR is applied for about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 minutes. In some embodiments, NIR is applied once, twice, three or more times.

In various embodiments, the method can further comprise administering an NIR-emitting device into a tumor, near a tumor, into a tumor resection cavity, near a resected tumor, or near a tumor cell. In some embodiments, the NIR-emitting device comprises an NIR-emitting light source. In one embodiment, administering the NIR-emitting device is via implanting the NIR device. In another embodiment, the NIR-emitting device is via a transcatheter procedure. In some embodiments, administering the NIR-emitting device comprises placing the NIR-emitting device on the tip of a catheter and using the catheter to administer the NIR-emitting device. In other embodiments, administering the NIR-emitting device comprises placing the NIR-emitting device as a balloon on the tip of a catheter and using the catheter to administer the NIR-emitting device.

Various embodiments of the present invention provide a system of treating, preventing, reducing the likelihood of having, reducing the severity of and/or slowing the progression of a condition in a subject. In various embodiments, the subject is a human. In various embodiments, the condition is a tumor or cancer. In certain embodiments, the condition is brain tumor, glioma, glioblastoma, and/or glioblastoma multiforme (GBM). The system may comprise or may consist of: a quantity of a nanoparticle and a means for applying NIR. In various embodiments, the system further comprises the instructions for using the nanoparticle and the means for applying NIR to treat, prevent, reduce the likelihood of having, reduce the severity of and/or slow the progression of the condition in the subject.

In various embodiments, the means for applying NIR is an NIR-emitting device. In some embodiments, the NIR-emitting device comprises an NIR-emitting light source. In various embodiments, the system further comprises a catheter. In one embodiment, the NIR-emitting device is placed on the tip of the catheter. In another embodiment, the NIR-emitting device is placed as a balloon on the tip of the catheter.

In various embodiments, the system may further comprise a quantity of a chemotherapeutic agent. In various embodiments, the nanoparticle and the chemotherapeutic agent are provided in one pharmaceutical composition. In various embodiments, the nanoparticle and the chemotherapeutic agent are provided in separate pharmaceutical compositions that may be administered together or individually. In one embodiment, the nanoparticle is conjugated to the chemotherapeutic agent. In another embodiment, the nanoparticle is loaded with the chemotherapeutic agent. In some embodiments, the nanoparticle is not conjugated to or loaded with the chemotherapeutic agent. In various embodiments, the system further comprises the instructions for using the nanoparticle, the chemotherapeutic agent and the means for applying NIR to treat, prevent, reduce the likelihood of having, reduce the severity of and/or slow the progression of the condition in the subject.

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof. In one embodiment, the nanoparticle is conjugated to the chemotherapeutic agent. In another embodiment, the nanoparticle is loaded with the chemotherapeutic agent. Still in another embodiment, the nanoparticle is not conjugated to or loaded with the chemotherapeutic agent. In various embodiments, the system further comprises the instructions for using the nanoparticle, the chemotherapeutic agent, and the means for applying NIR to treat, prevent, reduce the likelihood of having, reduce the severity of and/or slow the progression of the condition in the subject.

Various methods, compositions and systems of the present invention find utility in the treatment of various conditions, including but not limited to tumor, particularly brain tumor, glioma, glioblastoma, and/or glioblastoma multiforme (GBM).

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with various embodiments of the invention, effects of CNTs on cytotoxicity and proliferation of glioblastoma cells. A. U251 glioma cells were treated with different doses of CNTs and evaluated after 72 hours using the cell death ELISA. Doses of CNTs less than 3 μg/mL were not cytotoxic; apoptosis (left panel; p<0.05; n=3) and necrosis (right panel; p<0.05; n=3). B. U251 glioma cells treated with CNTs (0.3-3 μg/mL) were evaluated for proliferation after 72 hours using Brdu incorporation assay; the decrease was not significant (n=2).

FIG. 2 depicts, in accordance with various embodiments of the invention, effects of sequential treatment of CNTs and NIR on hyperthermia and CNT internalization by tumor cells. A. U251 glioma cells treated with CNTs (3 μg/mL) and NIR (6.75 W/cm²) (top line) demonstrated an increase in temperature of the media compared to NIR alone (***p<0.0001; n=3). Dashed line represents hyperthermia threshold (40° C.). B. U251 glioma cells were incubated with fluorescein-labeled CNTs (green); maximum internalization was observed after 24 hours. C. Astrocytes were cultured with fluorescein-labeled CNTs for 24 hours; few cells incorporated CNTs. Total numbers of cells were identified by Hoechst nuclear staining (blue).

FIG. 3 depicts, in accordance with various embodiments of the invention, the sequential treatment of CNTs and NIR induces lethal hyperthermia. U251 glioma cells were treated with CNTs (0.3, 1 and 3 μg/mL) and NIR once (A) or twice (B) for 5, 10 or 15 minutes; after 72 hours the MTT cell viability assay showed that exposure to NIR (6.75 W/cm²) reduced cell survival (n=3, *p<0.05, ***p<0.001 relative to 0 minutes of NIR of each condition). C. U251 glioma cells treated with CNTs (3 μg/mL) and NIR (10 minutes at 6.75 W/cm²) for 72 hours exhibit necrotic cell death as show by propidium iodide uptake (PI, red).

FIG. 4 depicts, in accordance with various embodiments of the invention, effects of CNTs and NIR on several different glioma cells compared to normal cells. A. GBM cell types (U251S, U251R, U87, U87R, LN229, LN229R, T98G), GBM cancer stem cells (USC02, USC04, USC08) and normal cells (human BEC and astrocytes) were treated with CNTs (3 μg/mL) and a single NIR treatment (10 minutes at 6.75 W/cm²). After 5 days cell viability was evaluated using the MTT assay. Normal cells demonstrated greater viability compared to tumor cells (n=3, ***p<0.001). B. U251 drug sensitive and drug resistant (U251R) cells were treated as described above. After 10 days the numbers of colonies were significantly reduced in CNTs (3 μg/mL) plus NIR (10 minutes at 6.75 W/cm²) treated cells (n=3, ***p<0.001); representative images of the colonies are depicted.

FIG. 5 depicts, in accordance with various embodiments of the invention, the sequential, combination therapy with CNTs and NIR inhibits tumor growth in vivo. Athymic nude mice implanted with U251R luciferase-positive cells were either left untreated or treated with NIR (10 minutes at 6.75 W/cm²) alone, CNTs alone (3 μg/mL), CNTs (3 μg/mL)+NIR and CNTs (0.3 μg/mL)+NIR; 4 animals/group. A. Tumor size was measured every 2 days. Control groups (untreated, treated with CNTs alone, or treated with NIR alone) were statistically identical. Only treatment with CNTs (3 μg/mL)+NIR showed tumor remission (n=4; *p<0.05, ***p<0.001 relative to untreated group). B. Representative images of mice at day 27 exhibited tumors. By contrast, animals treated with CNTs (3 μg/mL)+NIR group did not show any tumor (n=4) even at the termination of the experiment (day 80).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of glioma, delay or slowing of glioma, and amelioration or palliation of symptoms associated with glioma.

As used herein, the term “administering,” refers to the placement an agent as disclosed herein into a subject by a method or route which results in at least partial localization of the agents at a desired site.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. As used herein, the term “invasive” refers to the ability to infiltrate and destroy surrounding tissue. Melanoma is an invasive form of skin tumor. As used herein, the term “carcinoma” refers to a cancer arising from epithelial cells. Examples of cancer include, but are not limited to, brain tumor, nerve sheath tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor.

“Conditions” and “disease conditions,” as used herein may include, but are in no way limited to any form of malignant neoplastic cell proliferative disorders or diseases. Examples of such disorders include but are not limited to cancer and tumor. Examples of cancer include, but are not limited to, brain tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer.

The term “sample” or “biological sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

As used herein, a “subject” means a human or animal. Usually the animal is a vertebrate such as a primate, rodent, domestic animal or game animal Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., brain tumors) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value.

A “nanoparticle” is a particle having one or more dimensions of the order of 100 nm or less. A nanoparticle can be made of a variety of materials, including but limited to, gold, selenium, copper, platinum, or carbon, or a combination thereof. A nanoparticle can take a variety of shapes, including but limited to, tube, rod, shell, cage, sphere, fiber, or wire, or a combination thereof. As examples, nanofibers are fibers with diameters less than 100 nanometers; nanowires are about 75 nm in diameter, and range from 1 μm to 10 microns in length; nanotube are cylindrical nanoscale structures with length-to-diameter aspect ratios of up to 132,000:1. These particles can be bare, or can be capped with carboxylic acid, conventional citrate, and/or a positively charged ligand. These capping agents can readily be replaced with covalent and charge chemistries.

Nanorods are one morphology of nanoscale objects. Their dimensions usually range 1-100 nm and their aspect ratios (length divided by width) usually range 3-5. Nanorods may be synthesized from metals or semiconducting materials or their combinations. A nanorod has two ends and a linear body between the two ends. The two ends are also called the transverse or shorter ends. Accordingly, the longitudinal surface of the linear body is also called the longitudinal or longer end. The cross section of the linear body can be shaped as a variety of shapes, examples of which include but are not limited to, sphere, rectangular prism, dumbbell, triangle, rectangle, hexagon, or octagon, or a combination thereof. The two ends and the linear body may be made of the same or different materials. An “end surface” as used herein refers to the total area of an end plus the 0-10% of the linear body adjacent to the end; as a nanorod has two end surfaces, a “longitudinal surface” as used herein refers to the remaining 80-100% area of the linear body between the two end surfaces.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Our approach to specificity is the use of carbon nanotubes (CNT) to regulate the levels of hyperthermia. CNTs possess particular electrical, optical and thermal properties generated by the arrangement of the carbon atoms in a three dimensional cylindrical nanostructure. These structures have strong optical absorptions in the near-infrared (NIR) range (700-1400 nm) and generate heat through the release of vibrational energy. This property can be utilized for the induction of hyperthermia. The use of NIR radiation as a trigger is a promising platform since it is safe radiation for the human body, thereby circumventing off-target toxicity. Photothermal therapy for cancer results in few side effects, limited invasiveness, and enhanced sensitivity of tumor cells to hyperthermia. Wavelengths in the NIR range have a great advantage for in vivo applications because they cover the tissue-transparency window of the light spectrum. Furthermore, the low absorbance of NIR by water and biological tissues provides a favorable platform to irradiate CNTs. Hence, the combination of CNTs and NIR appears to be an effective therapy for glioblastoma treatment.

We use carbon nanotubes (CNT) coupled with near infrared radiation (NIR) to induce hyperthermia, as a novel non-ionizing radiation treatment for primary brain tumors, glioblastoma multiforme (GBM). In this invention, we report the therapeutic effect of hyperthermia-induced thermal ablation using the sequential administration of carbon nanotubes and NIR. In vitro studies were performed using glioma tumor cell lines (U251, U87, LN229, T98G). Glioma cells were incubated with CNTs for 24 hours followed by exposure to NIR for 10 minutes. Glioma cells preferentially internalized CNTs, which upon NIR exposure, generated heat, causing necrotic cell death. There were minimal effects to normal cells, which correlate to their minimal uptake of CNTs. Furthermore, this protocol caused cell death to glioma cancer stem cells, and drug-resistant as well as drug-sensitive glioma cells. This sequential hyperthermia therapy was effective in vivo, in the rodent tumor model resulting in tumor shrinkage and no recurrence after only one treatment. Therefore, this sequence of selective CNT administration followed by NIR activation provides a new approach to the treatment of glioma, particularly drug-resistant gliomas.

One embodiment of the present invention is the sequential use of carbon nanotubes and NIR to induce localized hyperthermia. NIR light-absorbing particles generate vibrational energy upon NIR radiation, which then induces heat. However, in order to use this approach in the clinic, an implanted NIR source may be preferred and no such medical device exists at the present time. The present invention offers the following novel features. (1) Specificity for glioblastoma: we demonstrated that CNTs are more selective to glioblastoma tumor cells and glioma stem cells, as compared to normal healthy cells. (2) The use of an implanted device for NIR administration: an NIR device enables the surgeon to control the power, time and site of irradiation based on tumor size and location.

Hyperthermia for cancer therapy faces several obstacles that restrict its clinical usage. However, the use of an implanted NIR device for the sequential treatment of CNTs and NIR offers key advantages: (1) Tumor-Specific heating: Hyperthermia only occurs in cells that have incorporated CNTs or other light-absorbing particles. Since CNTs show specificity for tumor cells, the heat generated by NIR exposure is tumor-specific and spare normal cells, compared to other types of hyperthermic therapies. Therapies may use MRI-guidance as a strategy to achieve tumor specificity; however, normal tissue surrounding the tumor may also be affected. (2) Minimally invasiveness: Biological tissues are fairly transparent to NIR wavelengths, meaning that NIR can cross tissues, evading the use of probes. Moreover, NIR beam aperture can be adjusted using lens, resulting in strict target focus.

### Treatment Methods and Systems

In various embodiments, the present invention provides a method of treating, preventing, reducing the likelihood of having, reducing the severity of and/or slowing the progression of a condition in a subject. The method comprises: providing a nanoparticle; administering a therapeutically effective amount of the nanoparticle to the subject; applying near infrared radiation (NIR) to the subject to induce hyperthermia, thereby treating, preventing, reducing the likelihood of having, reducing the severity of and/or slowing the progression of the condition in the subject.

In various embodiments, the condition is tumor. In various embodiments, the condition is a solid tumor including but are not limited to brain tumor, glioma, glioblastoma, and glioblastoma multiforme (GBM). In certain embodiments, the condition is chemoresistant irradiated brain tumor. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor.

In various embodiments, the subject is a human. In various embodiments, the subject is a mammalian subject including but not limited to human, monkey, ape, dog, cat, cow, horse, goat, pig, rabbit, mouse and rat.

In various embodiments, the nanoparticle is a nanotube, nanorod, nanoshell, nanocage, nanosphere, nanofiber, or nanowire, or a combination thereof. In various embodiments, the nanoparticle is made of carbon, gold, selenium, copper, platinum, or a combination thereof.

In various embodiments, the nanoparticle is conjugated to a chemotherapeutic agent. In various embodiments, the nanoparticle is loaded with a chemotherapeutic agent. In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

Typical dosages of an effective amount of the nanoparticle can be in the ranges recommended by the manufacturer where known therapeutic compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

In various embodiments, the nanoparticle may be administered once a day (SID/QD), twice a day (BID), three times a day (TID), four times a day (QID), or more, so as to administer an effective amount of the nanoparticle to the subject, where the effective amount is any one or more of the doses described herein.

In various embodiments, the nanoparticle is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 ng per mm³ tumor. In various embodiments, the nanoparticle is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 μg.

In various embodiments, the nanoparticle is administered once, twice, three or more times. In some embodiments, the nanoparticle is administered 1-3 times per day, 1-7 times per week, or 1-30 times per month. In various embodiments, the nanoparticle may be administered, for example, daily, weekly, biweekly, every fortnight and/or monthly at the aforementioned dosages. Still in some embodiments, a subject receives the nanoparticle for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In certain embodiments, the nanoparticle is administered to a human.

In accordance with the invention, the nanoparticle may be administered using the appropriate modes of administration, for instance, the modes of administration recommended by the manufacturer for each of the nanoparticle. In accordance with the invention, various routes may be utilized to administer the nanoparticle of the claimed methods, including but not limited to intratumoral, intracranial, intraventricular, intrathecal, epidural, intradural, aerosol, nasal, oral, transmucosal, transdermal, parenteral, implantable pump, continuous infusion, topical application, capsules and/or injections. In various embodiments, the nanoparticle is administered intratumorally, intracranially, intraventricularly, intrathecally, epidurally, intradurally, intravascularly, intravenously, intraarterially, intramuscularly, subcutaneously, intraperitoneally, orally, intranasally, or via inhalation. In further embodiments, the nanoparticle is administered with food or without food.

One of ordinary skill in the art would understand how to choose different parameters such as nanotube type, size, shape, presence of impurities, and route of administration. The CNTs used in our in vivo study did not reveal any toxicity.

In various embodies, the nanoparticle is provided in a pharmaceutical composition. In accordance with various embodiments, the pharmaceutical composition can further comprise a pharmaceutically acceptable excipient. In accordance with various embodiments, the pharmaceutical composition can further comprise a pharmaceutically acceptable carrier. In various embodiments, the pharmaceutical composition is formulated for intratumoral, intracranial, intraventricular, intrathecal, epidural, intradural, intravascular, intravenous, intraarterial, intramuscular, subcutaneous, intraperitoneal, oral, intranasal or via inhalation administration.

In various embodiments, NIR is applied after administering the nanoparticle. In various embodiments, NIR is applied about 1-10, 10-20, 20-30, 30-40, 40-50, 50-60 seconds, 1-10, 10-20, 20-30, 30-40, 40-50, 50-60 minutes, 1-3, 3-6, 6-9, 9-12, 12-15, 15-18, 18-21, 21-24 hours, 1-7 days, 1-4 weeks, 1-12 months, or 1-5 years after administering the nanoparticle. In one embodiment, NIR is applied to a tumor that has internalized a nanoparticle.

In various embodiments, NIR is applied at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 W/cm². In various embodiments, NIR is applied for about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 minutes.

In various embodiments, NIR is applied once, twice, three or more times. In some embodiments, NIR is applied 1-3 times per day, 1-7 times per week, or 1-30 times per month. In various embodiments, NIR may be applied, for example, daily, weekly, biweekly, every fortnight and/or monthly at the aforementioned dosages. Still in some embodiments, the subject receives NIR for 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In certain embodiments, NIR is applied to a human.

In various embodiments, the method further comprises administering an NIR-emitting device into a tumor, near a tumor, into a tumor resection cavity, near a resected tumor, or near a tumor cell. In accordance with the present invention, the NIR-emitting device comprises an NIR-emitting light source. In various embodiments, administering the NIR-emitting device is via implanting the NIR device.

In various embodiments, administering the NIR-emitting device is via a transcatheter procedure. In some embodiments, administering the NIR-emitting device comprises placing the NIR-emitting device on the tip of a catheter and using the catheter to administer the NIR-emitting device to a target location (e.g., into a tumor, near a tumor, into a tumor resection cavity, near a resected tumor, or near a tumor cell) using the catheter. In other embodiments, administering the NIR-emitting device comprises placing the NIR-emitting device as a balloon on the tip of a catheter and using the catheter to administer the NIR-emitting device to a target location. In accordance with present invention, the NIR-emitting device may emit NIR as a point source from the tip of a catheter or as a uniform distribution from a balloon on the tip of a catheter. In some embodiments, the NIR-emitting device remains connected to the catheter. In other embodiments, the NIR-emitting device is disconnected from the catheter and hence implanted in the subject after the catheter is retrieved. In one embodiment, the NIR-emitting device can be connected to an outside control unit via the catheter. In another embodiment, the NIR-emitting device can communicate wirelessly with an outside control unit. In various embodiments, the outside control unit can be connected to a power source and can be triggered to delivery NIR on a scheduled basis.

In various embodiments, the present invention also provides a system of treating, preventing, reducing the likelihood of having, reducing the severity of and/or slowing the progression of a condition in a subject. The system comprises: a quantity of a nanoparticle and a means for applying NIR. In accordance with the present invention, a therapeutically effective amount of the nanoparticle is administered to the subject and the means for applying NIR is used to apply NIR to the subject to induce hyperthermia. One non-limiting example of the means for applying NIR is an implantable NIR device. This implanted NIR device enables the surgeon to control the power, time and site of irradiation based on tumor size and location. The implanted device can be placed as a balloon catheter on the tip of a silicone catheter. The NIR light emitting source will be from the tip of the catheter. The light can be emitted as a point source from a catheter, or as a more uniform distribution via a balloon. The catheter can be connected to a power source which can be triggered remotely to deliver NIR on a scheduled basis. The implantable device can comprise laser, diodes, LED, LCD or other sources or radiation.

In various embodiments, the means for applying NIR is an NIR-emitting device. In accordance with the present invention, the NIR-emitting device comprises an NIR-emitting light source. In some embodiments, the device is configured for implantation. Still in some embodiments, the device is configured for administration or implantation to the subject via a transcatheter procedure. In some embodiments, the system further comprises a catheter, and the NIR-emitting device is placed on the tip of a catheter for administration to a target location (e.g., into a tumor, near a tumor, into a tumor resection cavity, near a resected tumor, or near a tumor cell). In other embodiments, the system further comprises a catheter, and the NIR-emitting device is placed as a balloon on the tip of a catheter for administration to a target location. In accordance with present invention, the NIR-emitting device may emit NIR as a point source from the tip of a catheter or as a uniform distribution from a balloon on the tip of a catheter. In some embodiments, the NIR-emitting device remains connected to the catheter. In other embodiments, the NIR-emitting device is disconnected from the catheter and hence implanted in the subject after the catheter is retrieved. In one embodiment, the system further comprises an outside control unit, and the NIR-emitting device may be connected to the outside control unit via the catheter, or may communicate wirelessly with the outside control unit. In various embodiments, the system further comprises a power source, and the outside control unit can be connected to the power source and can be triggered to delivery NIR on a scheduled basis.

The exact nature of the components configured in the inventive system depends on its intended purpose. In one embodiment, the system is configured particularly for the purpose of treating mammalian subjects. In another embodiment, the system is configured particularly for the purpose of treating human subjects. In further embodiments, the system is configured for veterinary applications, treating subjects such as, but not limited to, farm animals, domestic animals, and laboratory animals.

In various embodiments, the system further comprises the instructions for using the nanoparticle and the means for applying NIR to treat, prevent, reduce the likelihood of having, reduce the severity of and/or slow the progression of the condition in the subject. “Instructions for use” typically include a tangible expression describing the technique to be employed in using the components of the system to affect a desired outcome. Optionally, the system also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the system can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the system, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual system components. Thus, for example, a package can be a glass vial used to contain suitable quantities of a composition as described herein. The packaging material generally has an external label which indicates the contents and/or purpose of the system and/or its components.

### Pharmaceutical Compositions

In various embodies, the nanoparticle is provided in a pharmaceutical composition. In accordance with the invention, the pharmaceutical composition useful in the treatment of disease in mammals will often be prepared substantially free of naturally-occurring immunoglobulins or other biological molecules. Preferred pharmaceutical compositions will also exhibit minimal toxicity when administered to a mammal.

In various embodiments, the pharmaceutical composition further comprises a chemotherapeutic agent. In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof. In one embodiment, the nanoparticle is conjugated to the chemotherapeutic agent. In another embodiment, the nanoparticle is loaded with the chemotherapeutic agent. Still in another embodiment, the nanoparticle is not conjugated to or loaded with the chemotherapeutic agent.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

In various embodiments, the pharmaceutical composition is administered 1-3 times per day, 1-7 times per week, or 1-30 times per month. In various embodiments, the pharmaceutical composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In accordance with the invention, the pharmaceutical composition may be formulated for intravenous, intramuscular, subcutaneous, intraperitoneal, oral or via inhalation administration. In various embodiments, the pharmaceutical composition may be administered once a day (SID/QD), twice a day (BID), three times a day (TID), four times a day (QID), or more, so as to administer an effective amount of the nanoparticle to the subject, where the effective amount is any one or more of the doses described herein.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

Our invention provides a treatment method and an implanted device to induce hyperthermia upon the sequential application of a light-absorbing particle, carbon nanotubes, and near infrared radiation (NIR). Photo-thermal therapy for cancer has advantages as a treatment strategy because it is selective for tumor cells, can be used repeatedly in previously radiated tissue, has few off-target effects, and is relatively non-invasive. Our results show that hyperthermia is highly efficient for the treatment of therapy-resistant gliomas. We use an implanted medical device that can be placed into a tumor resection cavity, emitting NIR light, for the purpose of activating the CNT which have been internalized by malignant glioma cells. Our invention enables the NIR source to be controlled manually and in a remote manner, to be shown only to the area to be treated, and not the whole brain or healthy parts of the brain. With this device, the tumor, or tumor bed after surgery, can be treated with CNTs (or other NIR light-absorbing particles) and the time of exposure with NIR controlled remotely with the described device, thereby controlling the levels of hyperthermia to be generated. Moreover, the treatment can be performed in cycles, enabling hyperthermia to be delivered on a repeated basis, if shown to be effective. The application of hyperthermia can be further extended to other types of cancer, including skin melanoma, or other solid tumors where CNTs can be applied to the surgical field and an implanted catheter for emitting NIR device can be placed.

In various embodiments, the methods, systems and compositions described herein may be used in conjunction with other therapies including but not limited to chemotherapy and/or radiation therapy.

### EXAMPLES

The following examples are offered for illustrative purposes only, and are not intended to limit the scope of the present invention in any way.

### Example 1

### Materials and Methods

### Cell Culture and Treatments

U251 temozolomide (TMZ)-sensitive cells (U251S), U251 TMZ-resistant (U251R), U87 glioma cells, U87 TMZ-resistant (U87R), LN229 glioma cells, LN229 TMZ-resistant (LN229R), T98G glioma cells were cultured in 10% fetal calf serum (FCS; Omega Scientific Inc., Tarzana, Calif.) in Dulbecco's Modified Eagle's Media supplemented (Corning, Santa Clara, Calif.) with 100 U/mL penicillin and 0.1 mg/mL streptomycin. Human brain endothelial cells (BEC) and astrocytes were cultured in RPMI 1640 growth media (Mediatech Inc., Manassas, Va.) supplemented with 100 ng/mL EC growth supplement (Millipore, Temecula, Calif.), 10 mmol/L N-2-hy-droxyethylpiperazine-N-2-ethanesulfonic acid (HEPES) (Invitrogen, Carlsbad, Calif.), 24 mmol/L sodium pyruvate (Invitrogen), 300 U heparin (Sigma-Aldrich, St. Louis, Mich.), 1× Minimum Essential Medium (MEM) vitamin solution (Invitrogen), 1×MEM nonessential amino acids (Mediatech Inc.), 1% penicillin/streptomycin (Invitrogen) and 10% FCS. Cancer stem cells (USC02, USC04 and USC08) were cultured in serum-free medium composed of Dulbecco's modified Eagle medium [(DMEM)/F12+GlutaMAX-I)] supplemented with 100 U/mL penicillin, 0.1 mg/mL streptomycin, 1% B27 supplement (Invitrogen), 20 ng/mL epidermal growth factor (EGF; PeproTech, Oak Park, Calif.), and 20 ng/mL basic fibroblast growth factor-2 (FGF)-2 (Peprotech). All cell lines were cultured in a humidified incubator at 37° C. and 5% CO₂. Glioma cell lines were originally purchased from American Type Culture Collection; TMZ-resistant cells were developed by serial passaging of tumor cells with increasing concentrations of TMZ ranging from 10 to 100 μM over a period of 6 months. TMZ-resistant cell lines were exposed to 100 μM TMZ every other week to insure TMZ resistance. CNTs were purchased from Nanointegris (Nanointegris, Menlo Park, Calif.). For cell treatments, CNTs were diluted in cell media as appropriate; cells were kept in culture in the presence of CNTs for 24 hours prior to NIR exposure.

### Fluorescein-Labeling of CNTs

Single walled CNTs from Sigma Aldrich were shortened and carboxylated by heating in acid to yield highly functionalized nanotubes with carboxylic acid groups around the open ends and at defect sites in the sidewalls. Oxidized SWCNTs (0.25 mg/ml) were reacted with fluorescein-5-thiosemicarbazide (FC) (1 mg/ml) and 1-ethyl-3-(3-dimethylaminopropyl)-carbodiimide (EDC) for 2 hours at room temperature. The mixture was filtered using a Microcon centrifugal filter column with a molecular weight cut-off of 100,000 (Millipore). The residue on the filter membrane was washed with PBS several times until there was no color in the filtrate. The residue was then collected using a spatula and dispersed in PBS by sonication at intensity 4 to make a suspension of SWCNT-FC of 0.25 mg/ml. The suspension was maintained at 4° C. until use.

### Cell Death Assay

Cells were seeded at a density of 500 per well in 96-well plates and allowed to adhere overnight; cells were then treated with increasing concentrations of CNTs (0.3-30 μg/mL) for 72 hours. Supernatants and attached cells were collected separately and analyzed for necrosis and apoptosis respectively using commercially available ELISA kit per the manufacturers' instructions (Cell Death Detection ELISA^{PLUS}, Roche Applied Science, Indianapolis, Ind.). For the propidium iodide (PI) incorporation assay, cells were incubated with PI for the remaining 20 minutes of the assay. Photos were taken using EVOS fl AMF-4306 AMG microscopes.

### Cell Proliferation Assay

Cells were seeded at a density of 1×10⁴ per well and grown for 24 hours on 10 mm glass coverslips sitting in 24-well plates. CNTs (0.3, 1 and 3 μg/mL) were added as appropriate. U251S cells were treated with 5-bromo-2′-deoxyuridine (BrdU) (50 μM; Sigma-Aldrich) for the remaining 2 hours of the assay. Cells were fixed with 4% paraformaldehyde and non-specific binding was prevented by incubating cells in 3% bovine serum albumin (BSA) and 0.1% Triton X-100 solution for 30 minutes, at RT. Cells were incubated overnight at 4° C. with mouse monoclonal anti-BrdU (1:50; Molecular Probes, Eugene, Oreg.), then washed with PBS, and incubated for 2 hours at RT with Alexa Fluor 594 donkey anti-mouse (1:200; Molecular Probes). For nuclear labeling, cell preparations were counterstained with Hoechst 33342 (2 μg/mL) (Molecular Probes Sigma-Aldrich) in PBS, for 5 minutes at RT and mounted in Dako fluorescent medium (Dakocytomation Inc., Carpinteria, Calif.). All images were captured using EVOS fl AMF-4306 AMG microscopes; positive cells were counted out of the total number of cells, from 5 independent fields per coverslip.

### Tetrazolium Dye (MTT) Assay

Cells (2,000 or 500 cells per well for 72 hours or 5 days MTT respectively) were seeded in 96-well plates and allowed to adhere overnight. CNTs were then added at different concentrations for 24 hours, and then exposed to NIR. MTT was performed 72 hours or 5 days after the sequential treatment. Untreated, CNTs alone or NIR alone were used as controls. MTT assay was conducted according to the manufacturer's protocol (EMD Chemical, Gibbstown, N.J.). Absorbance was measured using the microtiter plate reader (Dynatech MR4000) at 490 nm.

### Colony Forming Assay (CFA)

Glioma cells were seeded in 12-well slide chambers (IBIDI, Verona, Wis.) at 200 cells per well and allowed to adhere overnight. Subsequently, cells were treated with CNTs for 24 hrs and then exposed to NIR. Cells were incubated for an additional 10 days. At the termination of the assay, colonies were visualized by staining with 1% methylene blue in methanol for 1 hour and quantified. Groups were plated in duplicate.

### In Vivo Studies

All animal protocols were approved by the Institutional Animal Care and Use Committee of the University of Southern California. Luciferase-labeled U251-TMZ-resistant glioma cells (5×10⁵ cells in 50 μL) were implanted subcutaneously. Fourteen days after implantation, the tumor was measured with calipers, mice were imaged, and randomly divided into groups of 4 each, and treatment was initiated as follows: untreated, treated with NIR alone (10 minutes at 6.75 W/cm²), CNTs alone (3 μg/mL), CNTs (3 μg/mL) plus NIR and CNTs (0.3 μg/mL) plus NIR. CNT treatment was performed by injecting intratumorally a total volume of 50 μL. One day after the CNT injection, animals were anesthetized and a single NIR laser treatment (10 minutes at 6.75 W/cm²) was performed in the site of the tumor. Tumor sizes were measured every 2 days using calipers and mice were imaged weekly. For the imaging, mice were injected with 1 mg/kg Viviren™ In Vivo Renilla Luciferase Substrate (Promega, Madison, Wis.) administered intravenously and imaged using the IVIS 200 optical imaging system (Caliper Life Sciences, Hopkinton, Mass.); images were analyzed using LIVING IMAGE software (Caliper Life Sciences).

### Example 2

### CNTs are not Cytotoxic and have Low Impact on Cell Proliferation

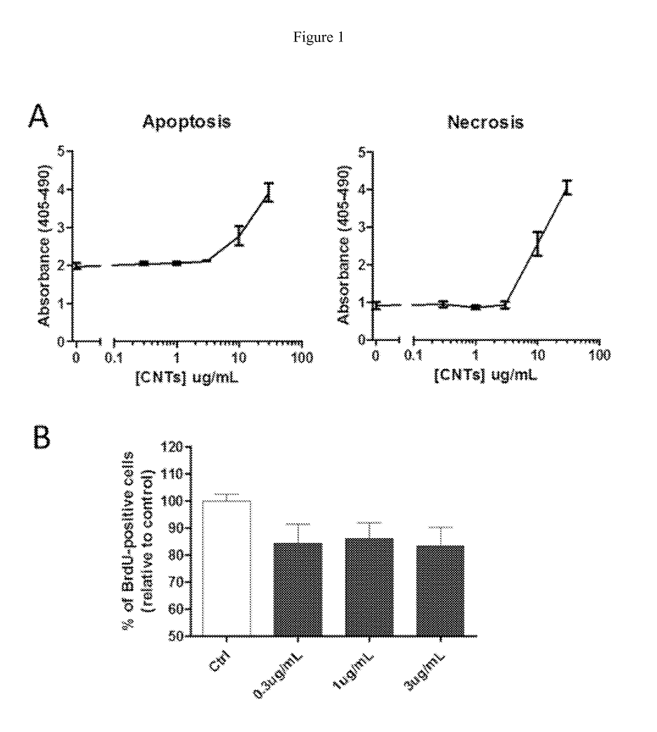

To determine whether exposure to CNTs alone induced cytotoxicity, U251 glioma cells were incubated with different concentrations of single-walled CNTs (0.3-30 μg/mL). Both apoptotic and necrotic cell death was evaluated 72 hours after treatment using a cell death ELISA kit. The results (FIG. 1A) show that CNTs did not induce either apoptosis or necrosis at concentrations equal to 3 μg/mL; higher doses of CNT demonstrated both apoptotic and necrotic cell death. Previous reports suggested that CNTs interact with filamentous actin (F-actin) monomers, causing a disruption of the cell cytoarchitecture and decreased proliferation. Therefore the effects of CNTs on cell proliferation were also tested. Using the BrdU assay, the results demonstrated that CNTs did not significantly inhibit cell proliferation, compared to untreated control cells (FIG. 1B). Based on these data 3 μg/mL was selected for further studies on the cytotoxic effects of the combination of CNTs and NIR.

### Example 3

### NIR-Exposed CNTs Induce Hyperthermia

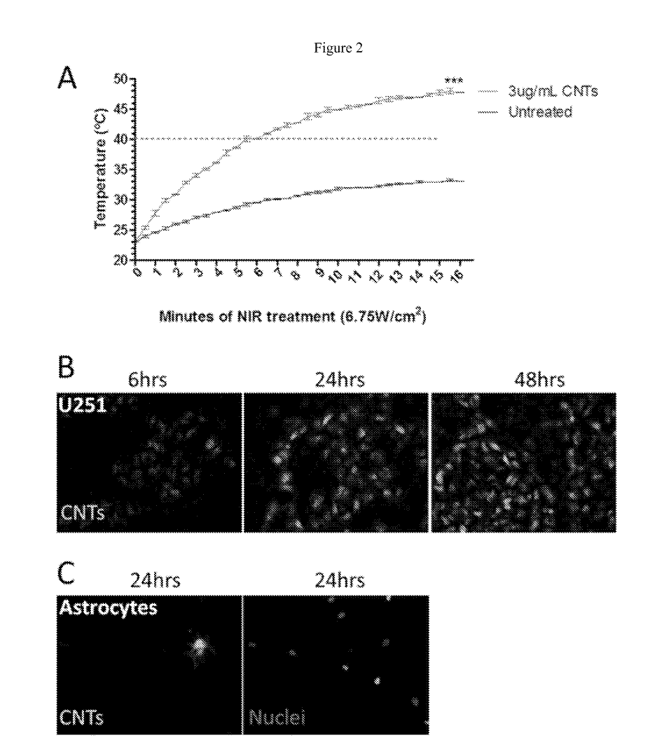

We next investigated whether the safe tolerable dose of CNTs (3 μg/mL) was sufficient to significantly increase the temperature upon NIR laser irradiation (6.75 W/cm²). The value of 6.75 W/cm² was selected since it was the highest power supported by the NIR laser used in this study, and therefore the most efficient in inducing hyperthermia. The hyperthermia threshold (40° C.) was reached after 5 minutes of NIR irradiation in the presence of 3 μg/mL CNTs, while the untreated aqueous solution never reached the 40° C. threshold, even after 15 minutes of exposure to NIR. At 15 minutes, the temperature of the culture media alone increased from 23° C. to 33.02±0.02° C.; by contrast in the presence of CNTs (3 μg/mL), the media significantly increased from 23° C. to 47.76±0.06° C. (FIG. 2A).

### Example 4

### CNTs are Preferentially Internalized by Tumor Cells

Since the internal concentration of CNTs may be critical to NIR susceptibility, the internalization kinetics were evaluated using fluorescently-labeled CNTs. U251 glioma cells were treated with fluorescently-labeled CNTs (3 μg/mL) and evaluated after 6, 24, and 48 hours of treatment (FIG. 2B). Optimal internalization was detected after 24 hours; longer incubation time did not increase the intracellular signal significantly. Control normal human astrocytes were also tested for their ability to internalize CNTs. Unlike U251 cells, few astrocytes internalized CNTs (1 per 100 cells counted) (FIG. 2C), indicating that CNTs are selectively internalized, with a preference for GBM cells as compared to normal astrocytes.

### Example 5

### Hyperthermia is Toxic to Glioma Cells

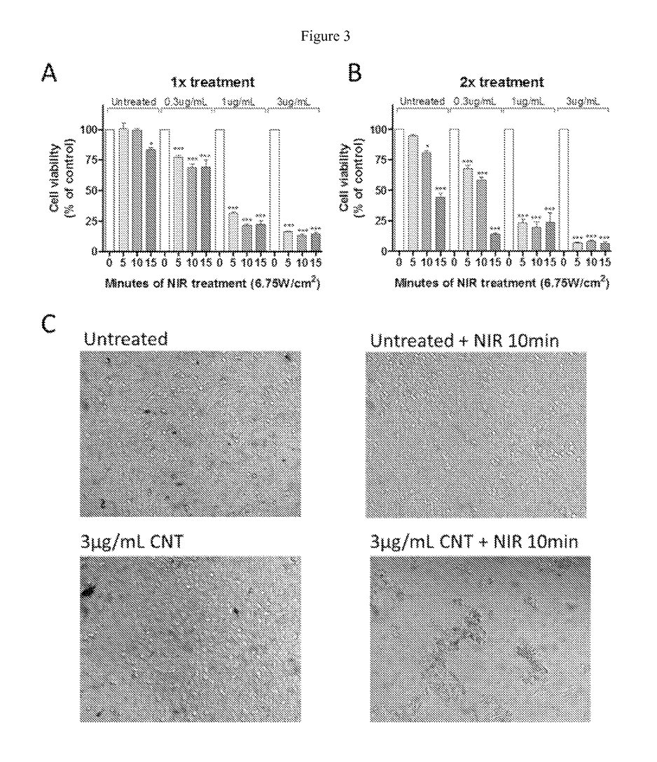

To evaluate the effects of hyperthermia on GBM, U251 cells were incubated with 0.3, 1 and 3 μg/mL of CNTs for 24 hours, subsequently an NIR laser (6.75 W/cm²) was shone for a pulse of 5, 10 or 15 minutes (FIG. 3A). The cell cultures were then evaluated after 72 hours using the MTT assay. The results show that 10 minutes at 3 μg/mL produced the maximum decrease in cell viability (13.38±0.83%). To determine whether a second exposure of CNT-treated cells to NIR would enhance cell death, NIR treatment was repeated for second exposure 24 hours after the first (FIG. 3B). Except for 15 minutes of exposure, which were also toxic to untreated (no CNTs) cells, there was no significant difference between one or two exposures to NIR treatment. Hyperthermia-induced cell death was dependent on both dose of CNTs and time of NIR exposure (FIGS. 3A & B).

All subsequent experiments presented in this study used the parameters of 24 hour treatment of CNTs (3 μg/mL) followed by a single 10 minutes NIR exposure (6.75 W/cm²). This selection was based on the fact that these settings have an NIR exposure that is safe for untreated cells (99.69±0.92% of control; FIG. 3A) but induce a higher cell death when combined with CNTs (13.38±0.83% of control; FIG. 3A). FIG. 3C depicts representative images of U251 glioma cells 72 hours after exposure to different treatments; subsequently cells were incubated with propidium iodide (PI), which labels (red) necrotic dead cells. Only in the presence of the combination of CNTs and NIR did the number of PI-positive cells increase, with the majority of PI-negative cells exhibiting a rounded morphology with multiple detached cells, indicative of cell stress. Although these cells were not PI-positive, their metabolism was likely compromised.

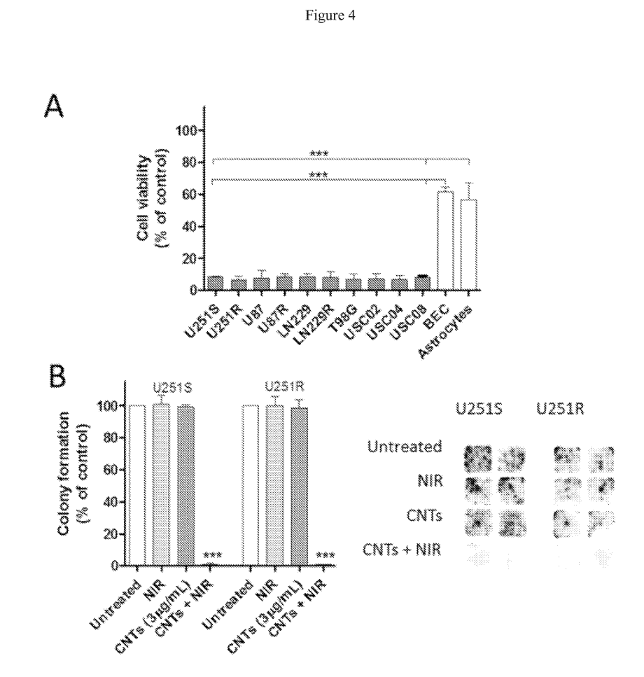

We next tested the long term effects of the combination of CNTs and NIR exposure on a variety of cells using the MTT assay for 7 days. Cell death was evaluated on the following cell cultures: U251 temozolomide (TMZ)-sensitive cells (U251S), U251 TMZ-resistant (U251R), U87 glioma cells, U87 TMZ-resistant (U87R), LN229 glioma cells, LN229 TMZ-resistant (LN229R), T98G glioma cells, human brain endothelial cells (BEC), human astrocytes and primary glioma cancer stem cells (GSC) isolated from three different human specimens (USC02, USC04, USC08) (FIG. 4A). The results showed that photo-induced hyperthermia is effective in killing different glioma cell lines, including GSC (U251S: 8.34±0.29%, U251R: 6.70±1.18%, U87: 7.70±2.77%, U87R: 8.70±2.02%, LN229: 8.53±1.13%, LN229R: 8.20±2.15%, T98G: 6.86±1.9%, USC02: 7.36±3.13%, USC04: 6.86±1.47%, USC08: 8.20±0.52%). Cell toxicity was achieved independently of TMZ-resistance status. By contrast, normal brain cells (BEC and astrocytes) exhibited no such cytotoxicity compared to the tumor cell populations (BEC: 61.49±2.95%, astrocytes: 56.77±10.57%). Thus in vitro studies showed that CNTs-induced hyperthermia is less cytotoxic to normal healthy brain cells, as compared to tumor cells. Overall, tumor cell survival in vitro was decreased to levels below 10% throughout all glioma-derived cell types that were tested (FIG. 4A). We also assessed the effects of this combination therapy on clonogenic survival using the colony forming assay (CFA), a long-term viability assay (10 days) that measures the ability of tumor cells to survive, proliferate, and form colonies. CNTs (3 μg/mL) and NIR laser alone (6.75 W/cm²; 10 minutes) had no statistically significant effects on colony-forming ability (FIG. 4B). However, the combination of CNTs and NIR exposure caused a reduction in viability to 0.926±0.93% in U251S and to 0.762±0.38% in U251R (P<0.0001).

### Example 6

### The Sequential Administration of CNTs and NIR Reduces Tumor Growth In Vivo

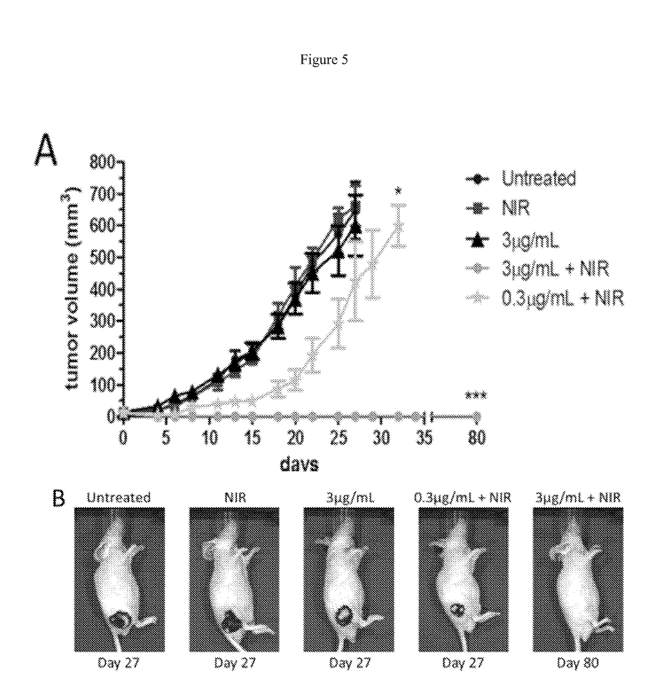

We next investigated the effects of CNT-induced hyperthermia in reducing tumor growth in vivo. Athymic nude mice were implanted with U251R renilla luciferase-positive cells into the hind flank. When tumors reached 12±2 mm³, animals were distributed into 5 experimental groups: untreated, NIR alone (10 min, 6.75 W/cm²); CNTs alone (3 μg/mL, 50 μL injected intratumorally); CNTs (3 μg/mL, 50 μL injected intratumorally)+NIR; CNTs (0.3 μg/mL, 50 μL injected intratumorally)+NIR. Twenty four hours after injection, tumors were exposed to NIR radiation (10 minutes, 6.75 W/cm²). Tumor growth was monitored and animals were euthanized when the tumor grew beyond 1.5 cm in diameter or the animals showed signs of stress or discomfort. The control groups of untreated, NIR alone-, CNTs alone-treated animals showed a similar tumor growth rate with no statistical differences (FIG. 5A). By contrast, a significant dose-dependent inhibition of tumor growth was obtained in animals treated with CNTs plus NIR. The effectiveness of this therapy was dose-dependent and more efficient at the highest concentration of CNTs tested (3 μg/mL), where a total elimination of the tumor was observed. Furthermore, there were no signs of tumor recurrence up to 80 days after treatment (FIG. 5B). Animals treated with a 10-fold lower concentration of CNTs (0.3 μg/mL) and NIR also exhibited a significant inhibition of tumor growth, however not as dramatic as that observed with 3 μg/mL CNTs in combination with NIR. These data demonstrate that sequential administration of CNT and NIR is effective in vivo and is suitable for patients with recurrent, drug-resistant gliomas.

In this study we demonstrate the efficacy of sequential administration of CNTs followed by NIR for the treatment of GBM. One embodiment of this invention is the sequential treatment for the production of localized hyperthermia. CNTs generate vibrational energy upon NIR radiation, which then induces heat. The novelty here is the demonstration that CNTs are relatively selective for glioblastoma tumor cells and stem cells, by contrast to normal healthy cells. The combination of CNTs and NIR radiation induces lethal hyperthermia to tumor cells independently of their chemoresistance status. Although temozolomide (TMZ) and radiation are routinely used in the clinic the GBM inevitably recurs when it becomes TMZ-resistant. (3) Here we show that photothermal therapy is effective on TMZ-resistant tumor cells. Therefore this therapy opens new avenues for the treatment of drug resistant, recurrent gliomas.

Without wishing to be bound by a particular theory, the mechanism of toxicity to tumor cells induced by CNTs may depend on different parameters such as nanotube type, size, shape, presence of impurities, and route of administration. The nanotubes used in our study were prepared by arc discharge and presented a 1.4 nm mean diameter and 1 μm mean length; these CNTs were non-toxic when used in concentrations up to 3 μg/mL. This range of concentrations induced a lower rate of cell proliferation, although the effects were not significant; higher doses of CNTs increased both apoptotic and necrotic cell death. Holt and colleagues reported that CNTs interact with filamentous actin (F-actin) monomers, disrupting the cell cytoarchitecture. Since cell division relies on these structural re-organizations, this suggests that CNTs affect tumor cell physiology.

Our data clearly show that CNTs, at non-toxic concentrations, generate heat when exposed to NIR in a time-dependent fashion (FIG. 2A). We also evaluated the internalization kinetics of fluorescein-labeled CNTs (FIG. 2B). The minimum time required for cells to achieve a maximum internalization was 24 hours. When cells were incubated for longer time periods, there was no incremental increase in intracellular fluorescence. Notably, we detected a preferential uptake of CNTs by glioma cells versus their healthy counterparts, astrocytes (FIGS. 2B and 2C). Differences in CNT uptake may be explained by the mechanism of internalization chosen by these cells. There are reports demonstrating that CNTs enter cells through endocytosis, specifically via tip recognition through receptor binding. These receptors may include scavenger receptors, lectin receptors and integrin receptors. Interestingly, up-regulated integrin signaling is common for several cancer types, including glioblastoma, thus supporting these observations. In order to select the optimal settings for our system, we treated glioma cells with a range of CNT doses at different NIR exposure times (FIGS. 3A and 3B). Ten minute exposure of cells to NIR was used routinely because at longer times (e.g., 15 minutes) cells without CNTs were sensitized and expressed a decrease in cell viability (FIG. 3B). Thus one NIR treatment of 10 minutes was sufficient, making this therapeutic modality a practical procedure for use in the clinic.

Single-walled CNTs induce hyperthermia by generating strong optical absorptions in the NIR region while biological tissues are fairly transparent to these wavelengths. A single 10 minute treatment showed a significant decrease in cell survival with no toxicity to untreated cells. NIR and CNT-induced cell death was predominately necrotic (FIG. 3C); hyperthermia is known to induce necrosis. Recently, the efficacy of CNTs and NIR was reported for different types of cancer. However, until our invention, it is still unclear if this strategy can be used for glioblastoma, especially for the chemotherapy-resistant phenotype. The effectiveness of hyperthermia was confirmed in several GBM cell lines. Five days after NIR exposure, the survival rate of all the cell lines tested was drastically decreased. Furthermore, this treatment was equally efficient on both therapy-resistant cells and glioma cancer stem cells, highlighting the clinical relevance of this therapy. We show here that GBM-derived cells were more sensitive to hyperthermia than normal BEC and astrocytes. These data are in accordance with previous studies which showed that selective tumor killing is achieved at temperatures between 40° C.-44° C., while most normal tissues remain undamaged at temperatures of up to 44° C. for as long as 1 hour.

The long term effects of this sequential treatment were evident for both U251S (TMZ-sensitive) and U251R (TMZ-resistant) glioma cells where these cells exhibited reduced survival of <1% ten days after treatment (FIGS. 4B and 4C). CNTs or NIR alone did not affect the number of colonies formed. Thus the sequential combination of these therapeutic moieties is crucial for maximum effectiveness in drug resistant cells.

As a proof of suitability for clinical use, we performed in vivo experiments using human TMZ-resistant glioma cells in the xenograft subcutaneous rodent tumor model. Drug-resistant cells were used because this is the most challenging malignant population to treat. In this mouse model, CNTs were delivered intratumorally. The following day the NIR laser exposure was initiated. A single 10 minute hyperthermia treatment was sufficient for the observed dramatic reduction in tumor size (FIGS. 5A and 5B). At the site of NIR treatment, animals pre-treated with CNTs generated a skin lesion which disappeared within 24-48 hours, leaving no visible mark on the skin. The depth of the lesion was related to the concentration of CNTs injected; this was a major consideration in determining the final dose of CNTs. All animals treated with this single sequential combination therapy at 3 μg/mL exhibited tumor shrinkage and ultimately no tumor. Furthermore the animals were followed for over 80 days with no detectable signs of tumor recurrence as confirmed by bioluminescence imaging. The effects observed are CNT concentration-dependent. Animals treated with a 10-fold lower CNT concentration (0.3 μg/mL) showed a significant tumor regression albeit not complete. These data are in accordance with our in vitro results and other publications where the effects of CNT and NIR treatment were concentration-dependent.

From a clinical standpoint, the use of hyperthermia in brain tumors has been called laser interstitial thermal energy; laser energy is directly transmitted to the tumor via a probe, inducing local hyperthermia in the brain tumor. The contour and extent of treatment is monitored via real time MRI imaging. Two companies, Monteris Medical (Plymouth, Minn.) and Biotex (Houston, Tex.) have both developed sophisticated high energy lasers for hyperthermia treatment. The problem with both systems is that local energy is deposited to a focal area using an introduced probe into the brain. As a result, each treatment requires a new invasive procedure. The use of high energy lasers also raises the risk of injury to normal cells in a non-targeted fashion. We demonstrate that our CNT treatment, followed by NIR, is selective, non-invasive, and repeatable. Specificity can be obtained by local introduction of CNT into the tumor cavity after resection. Only remaining tumor cells and tumor stem cells will incorporate CNTs. Since drug-resistant tumor cells also incorporate CNTs, hyperthermia-induced thermal ablation can overcome phenotypic diversity, and eliminate these cells. Non-invasive treatment can be obtained by using external NIR directed stereotactically into the tumor bed. In order to maximize the introduction of NIR, the bone flap from the craniotomy will be removed and placed intra-abdominally. Lastly, repeatability is most likely possible because NIR can be used multiple times, as long as CNTs remain in the tumor bed, and are taken up by existing tumor cells after the previously irradiated tumor cells die.

It is shown that NIR is capable of penetrating the skull. This skull penetration by NIR would be the ideal scenario. One alternative would be to leave the bone flap off, and place it intra-abdominally. Also, the advantage of leaving CNTs in the tumor bed enables NIR application on a repeated basis.

In conclusion, the sequential combined therapy using CNTs and then NIR is a powerful cancer therapy for the elimination of malignant cells with minimal effects on normal tissues. Furthermore, all GBM cells tested internalized CNTs and were sensitive to hyperthermia, independent of their drug resistance status. Our approach provides promising treatments of therapy-resistant gliomas and possibly other cancer types.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of treating, preventing, reducing the likelihood of having, reducing the severity of and/or slowing the progression of a condition in a subject, comprising:
providing a nanoparticle;
administering a therapeutically effective amount of the nanoparticle to the subject;
applying near infrared radiation (NIR) to the subject to induce hyperthermia, thereby treating, preventing, reducing the likelihood of having, reducing the severity of and/or slowing the progression of the condition in the subject.

2. The method of claim 1, wherein the condition is brain tumor, glioma, glioblastoma, and/or glioblastoma multiforme (GBM).

3. The method of claim 1, wherein the nanoparticle is a nanotube, nanorod, nanoshell, nanocage, nanosphere, nanofiber, or nanowire, or a combination thereof.

4. The method of claim 1, wherein the nanoparticle is made of carbon, gold, selenium, copper, platinum, or a combination thereof.

5. The method of claim 1, wherein the nanoparticle is administered intratumorally, intracranially, intraventricularly, intrathecally, epidurally, intradurally, intravascularly, intravenously, intraarterially, intramuscularly, subcutaneously, intraperitoneally, orally, intranasally, or via inhalation.

6. The method of claim 1, wherein the nanoparticle is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 ng per mm³ tumor.

7. The method of claim 1, wherein the nanoparticle is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 μg.

8. The method of claim 1, wherein the nanoparticle is administered once, twice, three or more times.

9. The method of claim 1, wherein the nanoparticle is provided in a pharmaceutical composition.

10. The method of claim 9, wherein the pharmaceutical composition further comprises a chemotherapeutic agent.

11. The method of claim 1, wherein NIR is applied at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 W/cm².

12. The method of claim 1, wherein NIR is applied for about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 minutes.

13. The method of claim 1, wherein NIR is applied once, twice, three or more times.

14. The method of claim 1, further comprising administering an NIR-emitting device into a tumor, near a tumor, into a tumor resection cavity, near a resected tumor, or near a tumor cell.

15. The method of claim 14, wherein the NIR-emitting device comprises an NIR-emitting light source.

16. The method of claim 14, wherein administering the NIR-emitting device is via implanting the NIR device.

17. The method of claim 14, wherein administering the NIR-emitting device is via a transcatheter procedure.

18. The method of claim 14, wherein administering the NIR-emitting device comprises placing the NIR-emitting device on the tip of a catheter and using the catheter to administer the NIR-emitting device.

19. The method of claim 14, wherein administering the NIR-emitting device comprises placing the NIR-emitting device as a balloon on the tip of a catheter and using the catheter to administer the NIR-emitting device.

20. A system for treating, preventing, reducing the likelihood of having, reducing the severity of and/or slowing the progression of a condition in a subject, comprising a quantity of a nanoparticle and a means for applying NIR.

21. The system of claim 20, further comprising a chemotherapeutic agent.

22. The system of claim 20, wherein the means for applying NIR is an NIR-emitting device.

23. The system of claim 22, wherein the NIR-emitting device comprises an NIR-emitting light source.

24. The system of claim 22, further comprising a catheter, wherein the NIR-emitting device is placed on the tip of the catheter.

25. The system of claim 22, further comprising a catheter, wherein the NIR-emitting device is placed as a balloon on the tip of the catheter.
